(19) United States
(12) Patent Application Publication
(10) Pub. No.: US 2008/0249017 A1
(43) Pub. Date: Oct. 9, 2008

(54) PHARMACEUTICAL FORMULATIONS OF GHRH MOLECULES

(75) Inventors: Helen Loughrey, Montreal (CA); Byeong Chang, Thousand Oaks, CA (US)

Correspondence Address: DARBY & DARBY P.C., P.O. BOX 770, Church Street Station, New York, NY, 10008-0770, US

(73) Assignee: THERATECHNOLOGIES INC., Ville Saint-Laurent (CA)

(21) Appl. No.: 12/098,298

(22) Filed: Apr. 4, 2008

Related U.S. Application Data
(60) Provisional application No. 60/909,985, filed on Apr. 4, 2007.

(51) Int. Cl. A61K 38/22; A61P 3/00; A61P 25/00

(52) U.S. Cl. 514/12

(57) ABSTRACT

Stabilized solid and liquid pharmaceutical formulations comprising a GHRH molecules as active ingredient, and more particularly GHRH analogs including [trans-3-hexenoyl]hGHRH (1-44) amide, are disclosed. The formulation comprises an anionic surfactant and a non-reducing sugar, and has a pH of about 4.0 to about 7.5. Also disclosed is the use of the formulation for the treatment of various conditions, methods of preparing the formulation, as well as kits containing it.

### CROSS-REFERENCE TO RELATED APPLICATIONS

This application claims the benefit, under 35 U.S.C. §119(e), of U.S. provisional application Ser. No. 60/909,985 filed on Apr. 4, 2007, which is incorporated herein by reference in its entirety.

### FIELD OF THE INVENTION

The present invention relates to pharmaceutical formulations, including liquid pharmaceutical formulations and solid pharmaceutical formulations, of GHRH molecules. Additionally, the present invention relates to methods for preparing such pharmaceutical formulations of a GHRH molecule, as well as uses thereof.

### BACKGROUND OF THE INVENTION

Growth hormone (GH) or somatotropin is secreted by the pituitary gland. Its activity is fundamental for the linear growth of a young organism but also for the maintenance of the integrity at its adult state. GH acts directly or indirectly on the peripheral organs by stimulating the synthesis of growth factors (insulin-like growth factor-I or IGF-I) or of their receptors (epidermal growth factor or EGF). The direct action of GH is of the type referred to as anti-insulinic, which favors the lipolysis at the level of adipose tissues. Through its action on IGF-I (somatomedin C) synthesis and secretion, GH stimulates the growth of cartilage and the bones (structural growth), protein synthesis and cellular proliferation in multiple peripheral organs, including muscles and skin. In adults, GH participates in the maintenance of a protein anabolism state and plays a primary role in the tissue regeneration phenomenon after a trauma.

The secretion of GH by the pituitary gland is principally controlled by two hypothalamic peptides, somatostatin and growth hormone-releasing hormone (GHRH; also known as growth hormone releasing factor or GRF). Somatostatin inhibits its secretion, whereas GHRH stimulates it.

Among all known GHRH molecules, GHRH analogs containing a hydrophobic tail as defined in the present application consist of modified versions or analogs of human GHRH that have been shown to have higher proteolytic stability in biological milieu and as a result, these analogs were shown to display longer duration of action resulting in enhanced growth hormone secretion and insulin like growth factor-1 synthesis (U.S. Pat. Nos. 5,861,379 and 5,939,386). Due to their superior plasma stability and pharmacological properties compared to the native GHRH (1-44) amide, these GHRH analogs were shown to confer therapeutic efficacy in several medical conditions, e.g., wasting associated with cystic fibrosis and COPD (International Application No. WO 05/037307), recovery after hip fracture, frailty in elderly population, enhancing immune response and HIV-associated lipodystrophy (U.S. Pat. No. 7,316,997).

In practical terms, it is very important to conserve the physical and chemical integrity a peptide or a protein compound of pharmaceutical interest during its manufacturing process, subsequent handling and storage. Loss of biological efficacy and potency has been associated with changes in physical (e.g., aggregation, denaturation, changes in secondary and higher order structures) and chemical (e.g., oxidation, deamidation, isomerization of individual amino acids) integrity.

Proteins and peptides are particularly prone to degradation at elevated temperatures. Lower temperatures generally decrease peptide/protein degradation. However, it is more economical to store the protein at room temperature, i.e., at about 20 to 25° C. In general, formulation stability is desirable for storage at either room temperature (at about 20° C. to about 25° C.) or refrigeration (at about 2° C. to about 8° C.).

There are also stability problems associated with manipulation during manufacture, with its long-term storage, and with manipulation prior to administration. Long-term storage can be achieved by freezing, freeze-drying (lyophilization), drying or dehydrating. These methods of long-term storage of biological proteins, impede degradation, aggregation, denaturation of native conformation, unfolding and/or nonspecific adsorption. However, the lyophilization process itself presents difficulties. As the volume of liquid decreases during the freezing process, the effective salt concentration increases dramatically, this may denature the protein and reduce the effective therapeutic activity upon reconstitution. In addition, formation of ice crystals during the freezing process may cause denaturation and also decrease the effective amount of bioactive peptide or protein available.

Some denaturation problems are specific to certain amino acids or to some amino acid sequence such as proteolysis, enzymatic degradation, oxidation, pH-related denaturation, etc. The amino acid sequence of GHRH is known to be subject to denaturations during long-term storage in liquid state and during lyophilization or other solidification processes.

Therefore, there is a need to provide improved formulations of GHRH molecules as well so as to improve retention of its bioactivity after long-term storage.

### SUMMARY OF THE INVENTION

The present invention relates to pharmaceutical formulations or compositions of a GHRH molecule, methods of preparation thereof, and uses thereof.

Accordingly, in an aspect, the present invention relates to a dried or solid pharmaceutical formulation comprising a GHRH molecule, an anionic surfactant, and a non-reducing sugar. In an embodiment, the formulation has a pH of about 4.0 to about 7.5 as measured upon suspension in water. In a further embodiment, the formulation has a pH of about 4.0 to about 7.0 as measured upon suspension in water. In an embodiment, the solid formulation is a lyophilized formulation. In an embodiment, the solid formulation is a dehydrated formulation. According to the present invention, the term “solid” includes, without limitation, lyophilized, dehydrated, frozen and any other solid forms.

The present invention also relates to a liquid pharmaceutical formulation comprising a GHRH molecule, an anionic surfactant, and a non-reducing sugar, and having a pH of about 4.0 to 7.5. In an embodiment, the formulation has a pH of about 4.0 to about 7.0.

The present invention further relates to a lyophilized or dehydrated pharmaceutical formulation prepared by lyophilizing or dehydrating the above-mentioned liquid formulation.

The liquid pharmaceutical formulation of the present invention is suitable for lyophilization or dehydration and provides a high stability of the GHRH molecule when the formulation is stored in a lyophilized, dried or solid form for a long period of time, such as at least 1 week, at least 2 weeks, at least 3 weeks, at least 1 month, at least 2 months, at least 3 months, at least 4 months or at least 6 months. The liquid pharmaceutical formulation of the present invention is suitable for lyophilization or dehydration and provides a high stability of the GHRH molecule when the formulation is stored in a lyophilized/dried form for a different temperature conditions, such as about 2° C. to about 8° C., about 20° C. to about 25° C., at about 40° C. or less than about 40° C.

According to an embodiment, the GHRH molecule is a GHRH analog of formula A:

X-GHRH Peptide (A)

wherein the GHRH peptide is a peptide of formula B:

(SEQ ID NO: 1)

A1-A2-Asp-Ala-Ile-Phe-Thr-A8-Ser-Tyr-Arg-Lys-A13-
Leu-A15-Gln-Leu-A18-Ala-Arg-Lys-Leu-Leu-A24-A25-
Ile-A27-A28-Arg-A30-R0 (B)

wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)n-CONH₂, with n=1 to 12; and
X is a hydrophobic tail anchored via an amide bond to the N-terminus of the peptide and the hydrophobic tail defining a backbone of 5 to 7 atoms;
wherein the backbone can be substituted by C₁₋₆ alkyl, C₃₋₆ cycloalkyl, or C₆₋₁₂ aryl and the backbone comprises at least one rigidifying moiety connected to at least two atoms of the backbone; said moiety is a double bond, triple bond, saturated or unsaturated C₃₋₉ cycloalkyl, or C₆₋₁₂ aryl.

In a further embodiment, X of formula A is:

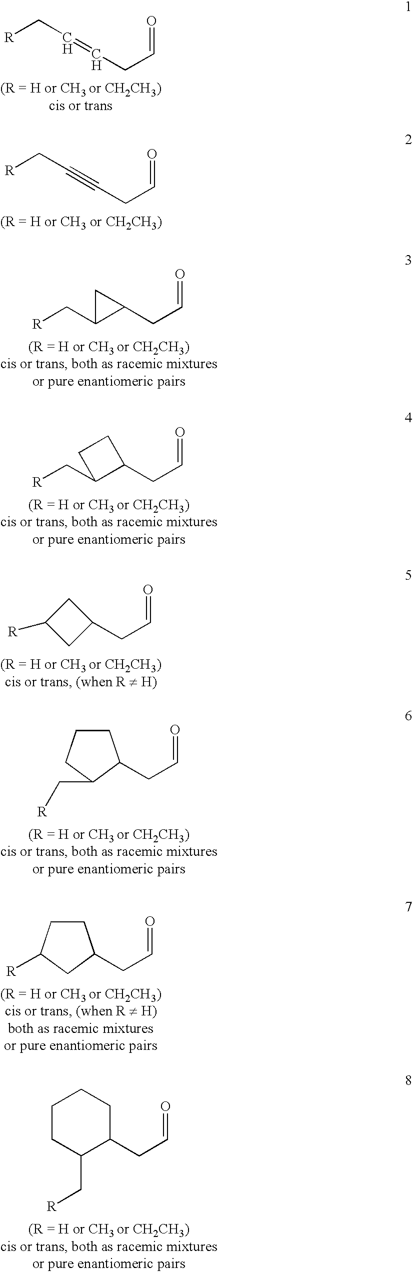

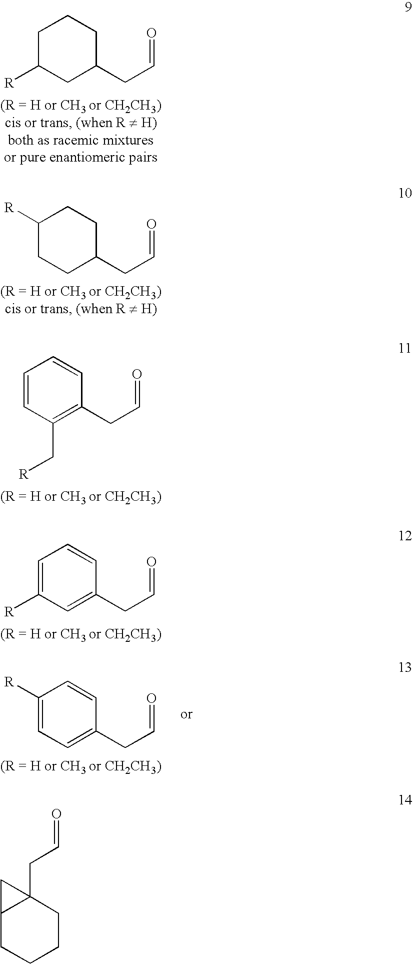

In an embodiment, A30 of formula B is:
(a) a bond;
(b) an amino acid sequence corresponding to positions 30-44 of a natural GHRH peptide (SEQ ID NO: 6), or
(c) the amino acid sequence of SEQ ID NO: 6 having a 1-14 amino acid deletion from its C-terminus.

In an embodiment, the GHRH peptide is:
(a) a polypeptide comprising the amino acid sequence of SEQ ID NO: 2 or 3;
(b) a polypeptide comprising the amino acid sequence of SEQ ID NO: 4 or 5; or
(c) the polypeptide of (a) having a 1 to 14 amino acid deletion from its C-terminus.

In an embodiment, the GHRH peptide is:
(a) a polypeptide having the amino acid sequence of SEQ ID NO: 2 or 3;
(b) a polypeptide having the amino acid sequence of SEQ ID NO: 4 or 5; or
(c) the polypeptide of (a) having a 1 to 14 amino acid deletion from its C-terminus.

In another embodiment, the GHRH analog is (hexenoyl trans-3)hGHRH(1-44)NH₂ (SEQ ID NO: 7).

In an embodiment, the above-mentioned solid formulation is a lyophilized formulation.

The concentration of GHRH molecule in the above-mentioned liquid formulation is not limited to a certain range. For example, the concentration of GHRH molecule in the above-mentioned liquid formulation may be between about 1 to about 20 mg/ml (e.g. 1, 2, 4, 6, 8, 10, 12, 14, 16, 18 or 20 mg/ml).

According to an embodiment of the invention, the non-reducing sugar of the solid formulation and/or the liquid formulation is trehalose or sucrose. The non-reducing sugar is preferably present in a stabilizing effective amount. In an embodiment, the non-reducing sugar is trehalose. In another embodiment, the non-reducing sugar is sucrose. In an embodiment, the non-reducing sugar is present in a concentration of about 0.1 to about 5% (w/w). In a further embodiment, the non-reducing sugar is present in a concentration of about 2% (w/w). The concentration of the non-reducing sugar and the concentration of any of the following constituents detailed herein correspond to the concentration in the liquid formulation or in the solution obtained from the suspension of the solid formulation.

According to an embodiment of the invention, the anionic surfactant of the solid formulation and/or the liquid formulation is a polyoxyethylene sorbitan alkyl ester. In a further embodiment, the polyoxyethylene sorbitan alkyl ester is polysorbate-20. In an embodiment, the anionic surfactant is present in an effective amount for preventing a surface-related stress. Surface-related stresses include, without limitation, aggregation, precipitation, unfolding and the like. In another embodiment, the surfactant is present in a concentration of about 0.001% (w/w) to about 0.1% (w/w). In further embodiment, the surfactant is present at a concentration of about 0.01% (w/w).

According to an embodiment of the present invention, the solid formulation and/or the liquid formulation optionally further comprise a bulking agent. In an embodiment, the bulking agent is present in an effective amount for providing a desired tonicity of the liquid formulation or the solution obtained from suspending the solid formulation. In an embodiment, the bulking agent is mannitol. In another embodiment, the bulking agent is present in a concentration of about 1 to about 10% (w/w). In a further embodiment, the bulking agent is present in a concentration of about 4% (w/w).

According to another embodiment of the present invention, the solid formulation and/or the liquid formulation optionally further comprise an anti-oxidant agent. In an embodiment, the anti-oxidant agent is methionine. In an embodiment, the anti-oxidant agent is present in an anti-oxidant effective amount. In another embodiment, the anti-oxidant agent is present in a concentration of about 0.1 mM to about 10 mM. In a further embodiment, the anti-oxidant agent is present in a concentration of about 1 mM.

According to an embodiment of the invention, the solid formulation and/or the liquid formulation is substantially free of polyethylene glycol. In a further embodiment, the solid formulation and/or the liquid formulation is free of polyethylene glycol. In an embodiment, the concentration of polyethylene glycol in the solid formulation and/or the liquid formulation is less than a stabilizing effective concentration. In an embodiment, the solid formulation and/or the liquid formulation contain less than about 0.1% (w/w) of polyethylene glycol. In another embodiment, the solid formulation and/or the liquid formulation contain less than about 0.01% of polyethylene glycol. In a further embodiment, the solid formulation and/or the liquid formulation contain less than about 0.001% (w/w) of polyethylene glycol.

According to an embodiment of the invention, the formulation has a pH of about 5.0 to about 6.0. According to another embodiment, the formulation has a pH of about 5.0. According to a further embodiment, the formulation has a pH of about 5.5. According to another further embodiment, the formulation has a pH of about 6.0. In an embodiment, the formulation further comprises a buffer. In another embodiment, the buffer is (i) succinate buffer, (ii) histidine buffer, (iii) phosphate buffer or (iv) any combination of (i) to (iii). In embodiments, the pH is of a liquid formulation prior to lyophilization or solidification, or of a liquid formulation prepared via suspension of a lyophilized or solid formulation into a liquid form.

According to an embodiment, the formulation comprises [trans-3-hexenoyl]hGHRH (1-44) amide, about 0.01% (w/w) of polysorbate-20, about 2% (w/w) of (i) trehalose, (ii) sucrose or (iii) any combination of (i) and (ii), about 4% (w/w) of mannitol; and a (i) succinate buffer, (ii) histidine buffer or (iii) any combination of (i) and (ii), wherein the formulation has a pH of about 5.0 to about 6.0.

According to a further embodiment, the formulation comprises [trans-3-hexenoyl]hGHRH (1-44) amide, about 0.01% (w/w) of polysorbate-20, about 2% (w/w) of sucrose, about 4% (w/w) of mannitol, and an histidine buffer, wherein the formulation has a pH of about 6.0.

According to a further embodiment, the formulation comprises [trans-3-hexenoyl]hGHRH (1-44) amide, about 0.01% (w/w) of polysorbate-20, about 2% (w/w) of sucrose, about 4% (w/w) of mannitol, and a succinate buffer, wherein the formulation has a pH of about 5.5.

According to a further embodiment, the formulation comprises [trans-3-hexenoyl]hGHRH (1-44) amide, about 0.01% (w/w) of polysorbate-20, about 2% (w/w) of sucrose, about 4% (w/w) of mannitol, and a succinate buffer, wherein the formulation has a pH of about 5.0.

According to a further embodiment, the formulation comprises [trans-3-hexenoyl]hGHRH (1-44) amide, about 0.01% (w/w) polysorbate-20, about 2% (w/w) trehalose, about 4% (w/w) mannitol, and a succinate buffer, wherein the formulation has a pH of about 5.5.

The invention further relates to the use of (a) the above-mentioned liquid pharmaceutical formulation, (b) a liquid pharmaceutical formulation prepared by the suspension of the above-mentioned solid pharmaceutical formulation with a sterile aqueous solution, or (c) a liquid pharmaceutical formulation prepared by the suspension of the lyophilized pharmaceutical formulation obtained by lyophilizing the above-mentioned liquid pharmaceutical formulation with a sterile aqueous solution, for the preparation of a medicament or as a medicament.

The invention further relates to the use of (a) the above-mentioned liquid pharmaceutical formulation, (b) a liquid pharmaceutical formulation prepared by the suspension of the above-mentioned solid pharmaceutical formulation with a sterile aqueous solution, or (c) a liquid pharmaceutical formulation prepared by the suspension of the lyophilized pharmaceutical formulation obtained by lyophilizing the above-mentioned liquid pharmaceutical formulation with a sterile aqueous solution, for the treatment of HIV-associated lipodystrophy, HIV-lipohypertrophy, GH deficiency, abdominal obesity, frailty, mild cognitive impairment, immune deficiency, wasting associated with a chronic disease, or malnutrition associated with a chronic disease.

The invention further relates to the use of (a) the above-mentioned liquid pharmaceutical formulation, (b) a liquid pharmaceutical formulation prepared by the suspension of the above-mentioned solid pharmaceutical formulation with a sterile aqueous solution, or (c) a liquid pharmaceutical formulation prepared by the suspension of the lyophilized pharmaceutical formulation obtained by lyophilizing the above-mentioned liquid pharmaceutical formulation with a sterile aqueous solution, for the preparation of a medicament for the treatment of at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease.

The invention also relates to the above-mentioned liquid pharmaceutical formulation, solid pharmaceutical formulation or lyophilized pharmaceutical formulation, for use in the treatment of at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease.

The invention also relates to a method of treating at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease, comprising the administration of (a) the above-mentioned liquid pharmaceutical formulation, (b) a liquid pharmaceutical formulation prepared by the suspension of the above-mentioned solid pharmaceutical formulation with a sterile aqueous solution, or (c) a liquid pharmaceutical formulation prepared by the suspension of the lyophilized pharmaceutical formulation obtained by lyophilizing the above-mentioned liquid pharmaceutical formulation with a sterile aqueous solution, to a subject.

A chronic condition includes, without limitation, HIV infection, AIDS, cystic fibrosis, chronic obstructive pulmonary disease, hip fracture, trauma, and major surgery.

In an embodiment, the sterile aqueous solution is sterile water or a sterile buffered solution having a pH between about 4.0 to about 7.5, in an embodiment a pH between about 4.0 to about 7.0, and in a further embodiment having a pH between about 5.0 to about 6.0.

According to an embodiment of the present invention, the liquid pharmaceutical formulation or suspended solid pharmaceutical formulation or lyophilized pharmaceutical formulation is administered by a subcutaneous, intramuscular, intravenous or intraperitoneal route.

The invention further relates to a kit or package for suspending a GHRH molecule formulation comprising the above-mentioned solid pharmaceutical formulation or lyophilized pharmaceutical formulation, in a sterile container. In an embodiment, the kit or package further comprises a sterile aqueous solution. In a further embodiment, the sterile aqueous solution is sterile water. In an embodiment, the kit or package further comprises instructions for suspending or reconstituting the solid pharmaceutical formulation to a liquid form.

The invention also relates to a method of preparing a stabilized pharmaceutical formulation of a GHRH molecule, comprising the steps of:

(a) combining a GHRH molecule, a non-reducing sugar and an anionic surfactant in an aqueous solution, thereby obtaining a liquid pharmaceutical formulation.

In an embodiment, the above-mentioned method of preparing a stabilized pharmaceutical formulation further comprises: (b) lyophilizing the liquid formulation of step (a).

In an embodiment, the above-mentioned stabilized pharmaceutical formulation of a GHRH molecule is stable for at least 1 week, at least 2 weeks, at least 3 weeks, at least 1 month, at least 2 months, at least 3 months, at least 4 months or at least 6 months. In an embodiment, the above-mentioned stabilized pharmaceutical formulation of a GHRH molecule is stable at different temperature conditions, such as about 2° C. to about 8° C., about 20° C. to about 25° C., at about 40° C. or less than about 40° C.

In another embodiment, the above-mentioned method of preparing a stabilized pharmaceutical formulation of a GHRH molecule further comprises the step (c) of suspending the lyophilized formulation with a sterile aqueous solution. In an embodiment, the sterile aqueous solution is sterile water.

Other objects, advantages and features of the present invention will become more apparent upon reading of the following non-restrictive description of specific embodiments thereof, given by way of example only with reference to the accompanying drawings.

The present description refers to a number of documents, the content of which is herein incorporated by reference in their entirety.

### BRIEF DESCRIPTION OF THE DRAWINGS

In the appended drawings:

FIG. 1 shows the effect of pH 4.0, 5.0 and 6.0 on the stability of [trans-3-hexenoyl]hGHRH (1-44) amide when lyophilized and stored at 40° C. (RP-HPLC data);

FIG. 2 shows the effect of the stabilizers: lactose, trehalose and sucrose, and the effect of methionine as anti-oxidant on the stability of [trans-3-hexenoyl]hGHRH (1-44) amide when lyophilized and stored at 40° C. (RP-HPLC data);

FIG. 3 shows the effect of the bulking agents: mannitol, glycine and PEG, on the stability of [trans-3-hexenoyl]hGHRH (1-44) amide when lyophilized and stored at 40° C. (RP-HPLC data);

FIG. 4 shows purity of [trans-3-hexenoyl]hGHRH (1-44) amide in different lyophilized formulations during storage at 40° C. (RP-HPLC data);

FIG. 5 shows purity of [trans-3-hexenoyl]hGHRH (1-44) amide in lyophilized formulations (F13 and F14) as compared to a non-stabilized formulation (5F644) stored at 4° C. over a period of 15 months (RP-HPLC data);

FIG. 6 shows the purity of [trans-3-hexenoyl]hGHRH (1-44) amide in lyophilized formulations (F13 and F14) as compared to a non-stabilized formulation (5F644) stored at 25° C. over a period of 15 months (RP-HPLC data); and

FIG. 7 provides the results of FT/IR analyses showing an overlaid comparison of the lyophilized formulations F4, F7 and F10 and the active principal ingredient (API) alone, in powder form and in liquid form (solubilized with water at 200 mg/ml).

### DESCRIPTION OF ILLUSTRATIVE EMBODIMENTS

The present invention provides pharmaceutical formulations comprising a GHRH molecule and more particularly, a GHRH analog of formula A detailed herein below. Several formulations of [trans-3-hexenoyl]hGHRH (1-44) amide have been exemplified and compared herein.

As used herein, “biologically acceptable” (or “pharmaceutically acceptable”) refers to materials characterized by the absence of (or limited) toxic or adverse biological effects in vivo. It refers to those compounds, formulations, formulations and/or dosage forms which are, within the scope of sound medical judgment, suitable for use in contact with the biological fluids and/or tissues and/or organs of a subject (e. g., human, animal) without excessive toxicity, irritation, allergic response, or other problem or complication, commensurate with a reasonable benefit/risk ratio.

The term “formulation” or “pharmaceutical formulation” as used herein refers to preparations which are in such form as to permit the active agents (e.g., a GHRH molecule, such as [trans-3-hexenoyl]hGHRH (1-44) amide) to be effective, and which contains no additional components which are toxic to the subjects to which the formulation would be administered. It refers to a formulation of the active agents (e.g., a GHRH molecule, such as [trans-3-hexenoyl]hGHRH (1-44) amide) and any buffers, bulking agents, adjuvants, carriers, stabilizers, surfactants and such other additives deemed necessary to maintain acceptable levels of activity and stability of the active agents during manufacture, storage, handling, and use. The pharmaceutical formulations of the present invention are suitable for lyophilization and the long-term storage of the active agents (e.g., a GHRH molecule, such as [trans-3-hexenoyl]hGHRH (1-44) amide) in a lyophilized form.

The term “GHRH molecule” as used in the context of the present invention includes, without limitation, human native GHRH (1-44) and fragments (1-40), (1-29), fragments ranging between 1-29 and the 1-44 sequence, and any other fragments; GHRH from other species and fragments thereof; GHRH variants containing amino acid(s) substitution(s), addition(s) and/or deletion(s) such that the amino acid sequence of the variant has at least about 90% of homology with the native amino acid sequence, in an embodiment at least about 95% of homology with the native amino acid sequence. In an embodiment, the above-mentioned fragments/variants retain at least about 10% of the activity of stimulating GH secretion as compared to the native GHRH; derivatives or analogs of GHRH or fragments or variants thereof having for a example an organic group or a moiety coupled to the GHRH amino acid sequence at the N-terminus, the C-terminus or on the side-chain; and salts of GHRH (human or from other species), as well as salts of GHRH fragments, variants, analogs and derivatives. The GHRH molecules of the present invention also encompass the GHRH molecules currently known in the art, including, without limitation, the albumin-conjugated GHRH (U.S. Pat. No. 7,268,113); pegylated GHRH peptide (U.S. Pat. Nos. 7,256,258 and 6,528,485); porcine GHRH (1-40) (U.S. Pat. No. 6,551,996); canine GHRH (U.S. patent application no. 2005/0064554); GHRH variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GHRHpeptide and variants thereof (U.S. Pat. No. 5,137,872).

The GHRH analogs include those described in U.S. Pat. Nos. 5,681,379 and 5,939,386, which also describe their method of synthesis. More particularly, these GHRH analogs are defined by the following formula A:

X-GHRH Peptide (A)

The GHRH peptide is a peptide of the following formula B: A1-A2-Asp-Ala-Ile-Phe-Thr-A8-Ser-Tyr-Arg-Lys-A13-Leu-A15-Gln-Leu-A18-Ala-Arg-Lys-Leu-Leu-A24-A25-Ile-A27-A28-Arg-A30-R0 (B) (SEQ ID NO: 1)

wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)n-CONH₂, with n=1 to 12.
   The group X is a hydrophobic tail anchored via an amide bond to the N-terminus of the peptide and the hydrophobic tail defining a backbone of 5 to 7 atoms. The backbone can be substituted by C₁₋₆ alkyl, C₃₋₆ cycloalkyl, or C₆₋₁₂ aryl and the backbone comprises at least one rigidifying moiety connected to at least two atoms of the backbone. The rigidifying moiety is a double bond, triple bond, saturated or unsaturated C₃₋₉ cycloalkyl, or C₆₋₁₂ aryl.

In an embodiment, group X is:

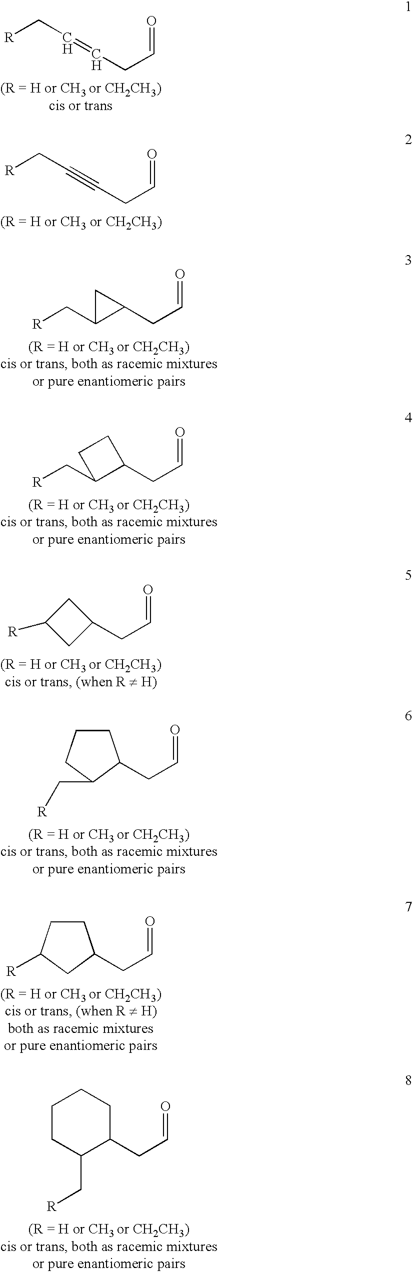

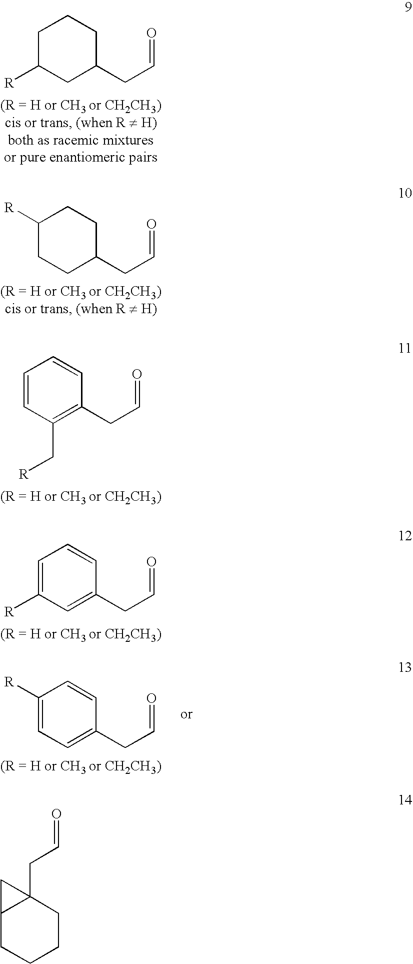

In an embodiment, in formula B, A30 is:
(a) a bond;
(b) an amino acid sequence corresponding to positions 30-44 of a natural GHRH peptide (SEQ ID NO: 6), or
(c) the amino acid sequence of (b) (SEQ ID NO: 6) having a 1-14 amino acid deletion from its C-terminus.

In an embodiment, the GHRH peptide is:
(a) a polypeptide comprising the amino acid sequence of SEQ ID NO: 2 or 3;
(b) a polypeptide comprising the amino acid sequence of SEQ ID NO: 4 or 5; or
(c) the polypeptide of (a) having a 1 to 14 amino acid deletion from its C-terminus.

In an embodiment, the GHRH peptide is:
(a) a polypeptide having the amino acid sequence of SEQ ID NO: 2 or 3;
(b) a polypeptide having the amino acid sequence of SEQ ID NO: 4 or 5; or
(c) the polypeptide of (a) having a 1 to 14 amino acid deletion from its C-terminus.

In an embodiment, the GHRH molecule is (hexenoyl trans-3)hGHRH(1-44)NH₂ (SEQ ID NO: 7). [trans-3-hexenoyl]hGHRH (1-44) amide (also referred to as (hexenoyl trans-3)hGHRH(1-44)NH₂) is a synthetic human growth hormone releasing factor analog that comprises the 44-amino acid sequence of human growth hormone releasing factor (hGHRH) on which a hexenoyl moiety, a C₆ side chain, has been anchored on Tyr1 at the N-terminus.

[trans-3-hexenoyl]hGHRH (1-44) amide has the following structure:

(SEQ ID NO: 7)

(trans)CH₃—CH₂—CH═CH—CH₂—CO-Tyr-Ala-Asp-Ala-Ile-
Phe-Thr-Asn-Ser-Tyr-Arg-Lys-Val-Leu-Gly-Gln-Leu-
Ser-Ala-Arg-Lys-Leu-Leu-Gln-Asp-Ile-Met-Ser-Arg-
Gln-Gln-Gly-Glu-Ser-Asn-Gln-Glu-Arg-Gly-Ala-Arg-
Ala-Arg-Leu-NH₂.

The term “solid” as used herein in the context of a formulation of the invention refers to the formulation in a form which is substantially free of moisture, e.g., a solid (e.g., powder) form. Such a solid formulation may be prepared by any method of moisture removal, e.g., by lyophilization, dehydration, or other drying methods.

The term “suspension” as used herein is intended to refer to suspension, resuspension, reconstitution and/or solubilisation depending on the context. For a matter of consistency, the term “suspension” is used herein to generally refer to the addition of a suitable liquid to the solid formulation.

The term “bulking agent” as used herein refers to a compound used to provide an adequate or desired tonicity of the solution resulting from the suspension of the solid formulation. Preferably, the adequate or desired tonicity of the solution is equal to or approximates isotonicity with physiological fluid of the subject to which the solution is administered. For example, one or more sugars may be used as the bulking agent. Sugars, as used herein, include, but are not limited to, monosaccharides, oligosaccharides and polysaccharides. Examples of suitable sugars include, but are not limited to, mannose, sorbose, xylose, maltose, lactose, sucrose, and dextran. Sugar also includes sugar alcohols, such as mannitol, inositol, dulcitol, xylitol and arabitol. Mixtures of sugars may also be used in accordance with this invention. In an embodiment, the bulking agent is mannitol. For example, one or more amino acids, such as glycine, may be used as the bulking agent. The bulking agent is in concentration of about 1 to about 10% (w/w) in the formulation. In an embodiment, the bulking agent is in concentration of about 3 to about 5% (w/w). In a further embodiment, the bulking agent is in concentration of about 4% (w/w).

In an embodiment, the pharmaceutical formulations of the present invention have a pH of about 4.0 to about 7.5. In a further embodiment, the pharmaceutical formulations of the present invention have a pH of about 4.0 to about 7.0. In a further embodiment, the pharmaceutical formulations of the present invention have a pH of about 5.0 to about 6.0. In a further embodiment, the pharmaceutical formulations of the present invention have a pH of about 6.0. In another embodiment, the pharmaceutical formulations of the present invention have a pH of about 5.5. In another embodiment, the pharmaceutical formulations of the present invention have a pH of about 5.0. In another embodiment, the pharmaceutical formulations of the present invention have a pH above 5.0.

In an embodiment, the formulations of the present invention further comprise a buffer. The suitable amount of buffer will vary depending on the type of buffer used and its buffering capacity. The buffer should be of a type appropriate to and present in the formulation in an amount sufficient to maintain the final pH of the formulation in the pH range mentioned above. In an embodiment, the buffer is sodium succinate (succinate). In another embodiment, the buffer is L-histidine (histidine). In another embodiment, the buffer is sodium phosphate (phosphate). These buffers are frequently available as a salt. In an embodiment, the concentration of buffer in the pharmaceutical formulations of the invention is from about 0.1 mM to about 50 mM. In another embodiment, the concentration of buffer in the pharmaceutical formulations of the invention is from about 1 mM to about 30 mM. In a further embodiment, the concentration of buffer in the pharmaceutical formulations of the invention is from about 5 mM to about 20 mM. In a further embodiment, the concentration of buffer in the pharmaceutical formulations of the invention is about 10 mM.

The amount of active principal ingredient (e.g., a GHRH molecule, such as [trans-3-hexenoyl]hGHRH (1-44) amide) contained in pharmaceutical formulations of the present invention can be determined depending on the nature and/or severity of the disease to be treated, the characteristics of the patient (age, weight, etc) and other factors. Generally, the pharmaceutical formulation of the invention comprises about 1 to about 40 000 μg/ml of active principal ingredient (e.g., a GHRH molecule, such as [trans-3-hexenoyl]hGHRH (1-44) amide). In an embodiment, the pharmaceutical formulation of the invention comprises about 1000 to about 8000 μg/ml (about 0.099% to about 0.792% by weight) of active principal ingredient (e.g., a GHRH molecule, such as [trans-3-hexenoyl]hGHRH (1-44) amide). In another embodiment, the pharmaceutical formulation of the invention comprises about 1000 to about 4000 μg/ml (about 0.099% to about 0.396% by weight) of active principal ingredient (e.g., a GHRH molecule, such as [trans-3-hexenoyl]hGHRH (1-44) amide). In a further embodiment, the pharmaceutical formulation of the invention comprises about 1000 μg/ml (about 0.099% by weight) of active principal ingredient (e.g., a GHRH molecule, such as [trans-3-hexenoyl]hGHRH (1-44) amide). In another embodiment, the pharmaceutical formulation of the invention comprises about 4000 μg/ml (about 0.396% by weight) of active principal ingredient (e.g., a GHRH molecule, such as [trans-3-hexenoyl]hGHRH (1-44) amide). In embodiments, the formulation comprises an amount of the active principal ingredient (e.g., a GHRH molecule, such as [trans-3-hexenoyl]hGHRH (1-44) amide) to effect administration of a dose of the active principal ingredient (e.g., a GHRH molecule, such as [trans-3-hexenoyl]hGHRH (1-44) amide which is greater than or equal to about 1 mg; in a further embodiment, about 1 mg; in a further embodiment, about 2 mg; in a further embodiment, greater than or equal to about 2 mg. At high concentration of the active principal ingredient (e.g., a GHRH molecule, such as [trans-3-hexenoyl]hGHRH (1-44) amide), the buffer may used at a higher concentration. For example, in the studies described herein, the pH of a formulation containing 10 or 30 mg/ml of [trans-3-hexenoyl]hGHRH (1-44) amide has been maintained with 30 mM of histidine buffer.

In an embodiment, the pharmaceutical formulations of the present invention may further comprise one or more surfactants. Typical examples of surfactants include:

A) nonionic surfactants, e.g., sorbitan fatty acid esters such as sorbitan monocaprylate, sorbitan monolaurate, sorbitan monopalmitate; glycerin fatty acid esters such as glycerin monocaprylate, glycerin monomyristate, glycerin monostearate; polyglycerin fatty acid esters such as decaglyceryl monostearate, decaglyceryl distearate, decaglyceryl monolinoleate; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate; polyoxyethylene sorbitol fatty acid esters such as polyoxyethylene sorbitol tetrastearate, polyoxyethylene sorbitol tetraoleate; polyoxyethylene glycerin fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol ether, polyoxyethylene polyoxypropylene propyl ether, polyoxyethylene polyoxypropylene cetyl ether; polyoxyethylene alkyl phenyl ethers such as polyoxyethylene nonyl phenyl ether; polyoxyethylene hardened castor oils such as polyoxyethylene castor oil, polyoxyethylene hardened castor oil (polyoxyethylene hydrogenated castor oil); polyoxyethylene beeswax derivatives such as polyoxyethylene sorbitol beeswax; polyoxyethylene lanolin derivatives such as polyoxyethylene lanolin; polyoxyethylene fatty acid amides such as polyoxyethylene stearic acid amide;

B) anionic surfactants, e.g., alkyl sulfates having a C₁₀₋₁₈ alkyl group such as sodium cetyl sulfate, sodium lauryl sulfate, sodium oleyl sulfate; polyoxyethylene alkyl ether sulfates such as sodium polyoxyethylene lauryl sulfate; alkyl sulfosuccinic acid ester salts having a C₈₋₁₈ alkyl group such as sodium laurylsulfosuccinate; and

C) natural surfactants, e.g., lecithin; glycerophospholipids; sphingophospholipids such as sphingomyelin; sucrose fatty acid esters of C₁₂₋₁₈ fatty acids. One or more of these surfactants may be added in combination to formulations of the present invention.

In an embodiment, the surfactant of the pharmaceutical formulations of the present invention is an anionic surfactant. In a further embodiment, the surfactant of the pharmaceutical formulations of the present invention is polyoxyethylene sorbitan alkyl ester. In yet a further embodiment, the surfactant of the pharmaceutical formulations of the present invention is Polysorbate-20 (T20 or Tween-20™).

In another embodiment, the amount of surfactant in the pharmaceutical formulations of the present invention is about 0.0001% to about 10% (w/w). In a further embodiment, the amount of surfactant in the pharmaceutical formulations of the present invention is about 0.001% to about 5% (w/w). In yet a further embodiment, the amount of surfactant in the pharmaceutical formulations of the present invention is about 0,01% (w/w).

In an embodiment, the pharmaceutical formulations of the present invention may further comprise one or more stabilizing agents or stabilizers. As used herein, the term “stabilizer” is intended to mean a compound used to stabilize the therapeutic agent against physical, chemical, and/or biochemical processes that would reduce the therapeutic activity of the agent. Suitable stabilizers are non-reducing sugars including, by way of example and without limitation, sucrose (or saccharose) and trehalose; and non-reducing polyols including, by way of example and without limitation, sorbitol, mannitol, maltitol, xylitol, glycol, glycerol and ethylene glycol. Commercial source of polyethylene glycol is not suitable as it often includes contaminants that may cause degradation of the GHRH molecule. In an embodiment, the above-mentioned formulations are substantially free of polyethylene glycol. In a further embodiment, the above-mentioned formulations are free of polyethylene glycol.

In an embodiment, the pharmaceutical formulations of the present invention comprise a non-reducing sugar. “Non-reducing sugar” as used herein refers to a sugar (e.g., a monosaccharide or polysaccharide) that does not contain a hemi-acetal, for example a carbohydrate or sugar characterized by having a glycosidic bond formed between the reducing ends of the sugar units, and not between a reducing end of one sugar unit and a non-reducing end of the other sugar unit. In a further embodiment, the above-mentioned non-reducing sugar is trehalose or sucrose. In a further embodiment, the above-mentioned non-reducing sugar is sucrose. In another further embodiment, the above-mentioned non-reducing sugar is trehalose. The non-reducing sugar is in a concentration of about 0.1 to about 5% (w/w) in the formulations of the invention. In an embodiment, the non-reducing sugar is in a concentration of about 1 to about 3% (w/w). In a further embodiment, the non-reducing sugar is in a concentration of about 2% (w/w).

In another embodiment, the non-reducing sugar is present in an amount of about 1% (w/w) to about 5% (w/w) in the pharmaceutical formulation. In a further embodiment, the non-reducing sugar is present in an amount of about 2% (w/w) in said formulation.

The pharmaceutical formulations of the present invention may further contain diluents, solubilizing agents, excipients, pH-modifiers, soothing agents, buffers, sulfur-containing reducing agents, antioxidants or the like, if desired. For example, sulfur-containing reducing agents include N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, methionine and sulfhydryl-containing compounds such as thioalkanoic acid having 1 to 7 carbon atoms. Antioxidants include methionine, erythorbic acid, dibutylhydroxytoluene, butylhydroxyanisole, α-tocopherol, tocopherol acetate, L-ascorbic acid and salts thereof, L-ascorbyl palmitate, L-ascorbyl stearate, sodium bisulfite, sodium sulfite, triamyl gallate, propyl gallate or chelating agents such as disodium ethylenediamine tetraacetate (EDTA), sodium pyrophosphate, sodium metaphosphate. Other components commonly added may also be contained, e.g., inorganic salts such as sodium chloride, potassium chloride, calcium chloride, sodium phosphate, potassium phosphate, sodium bicarbonate; and organic salts such as sodium citrate, potassium citrate, sodium acetate.

A stable formulation is one in which the active principal ingredient, i.e. the GHRH molecule (e.g., [trans-3-hexenoyl]hGHRH (1-44) amide) therein essentially retains its physical and chemical stability and integrity upon storage. Various analytical techniques for measuring protein or peptide stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept, for example, at about 40° C. for 2 weeks to 1 month (and for up to 6 months), at which time stability is measured. The formulation may also be kept, for example, at about 2° C. to about 8° C. (e.g., about 4° C.) or in ambient room temperature conditions (about 15° C. to about 30° C., preferably about 20° C. to about 25° C.) for at least 6 months, at which time stability is measured. The formulation of the present invention offers a better stability of the GHRH molecule in its liquid or solid form and is also suitable for preserving the stability of the GHRH molecule in solid or lyophilized form for a period of storage at elevated temperature (e.g. 40° C.), at room temperature (i.e. 20-25° C.), at refrigerated temperature (i.e. 2-8° C.). The period of storage may for example be expressed in weeks, months or years, and may be at least 1 week, at least 2 weeks, at least 4 weeks, at least 6 weeks, at least 8 weeks, at least 3 months, at least 4 months or at least 6 months. For example, a “stable” formulation may be one wherein more than about 80%, more than about 90%, more than about 95%, more than about 96%, more than about 97%, more than about 98%, or more than about 99% of the non-degraded active agent is present in the formulation. The stability of the formulations of the present invention may be measured using RP-HPLC (e.g., see Examples below). A “stabilizing effective amount or concentration” as used herein is meant to designate an amount or concentration effective to obtain a stable formulation wherein more than about 80%, more than about 90%, more than about 95%, more than about 96%, more than about 97%, more than about 98%, or more than about 99% of the non-degraded active agent is present in the formulation.

The formulations of the invention are useful as a medicament, for therapeutic applications, for example for the treatment of lipodystrophy (e.g. HIV-related lipodystrophy), lipohypertrophy, GH deficiency, abdominal obesity, dyslipidemia, hypertriglyceridemia, syndrome X, improvement in quality of life, frailty, daytime vigilance, mild cognitive impairment (or cognitive function) including thinking, reasoning, problem solving and memory, immune deficiency, muscular wasting associated with a chronic or long-term disease, or malnutrition associated with a chronic or long-term disease. A chronic or long-term disease includes, without limitation, HIV infection, AIDS, cystic fibrosis, chronic obstructive pulmonary disease, hip fracture, trauma, and major surgery. In an embodiment, the formulation of the present invention is useful for the treatment of HIV-related lipodystrophy. In another embodiment, the formulation of the present invention is useful for the treatment of chronic obstructive pulmonary disease. In another embodiment, the formulation of the present invention is useful for the treatment of cystic fibrosis.

As used herein, the terms “subject” or “patient” are taken to mean warm blooded animals such as mammals, for example, cats, dogs, mice, guinea pigs, horses, bovine cows, sheep and humans. In an embodiment, the subject is a mammal. In a further embodiment, the above-mentioned subject is a human.

The term “treatment” as used herein, is defined as the application or administration of a therapeutic agent to a subject, or application or administration of a therapeutic agent to an isolated tissue or cell line from a subject, who has a disorder, a disease, a symptom of disorder or disease, or a predisposition toward a disorder or disease, with the purpose to cure, heal, alleviate, delay, relieve, alter, remedy, ameliorate, improve or affect the disorder/disease, the symptoms of disorder/disease or the predisposition toward disorder/disease.

The invention further provides a method to prepare the formulations described herein. The method comprises formulating or combining together (e.g., dissolving, mixing) the ingredients under conditions to obtain the desired formulation (e.g., with respect to formulation, concentration, pH, etc.). For example, with respect to pH, the pH of the formulation may be determined and adjusted accordingly (if necessary) to be within the desired range. Examples of such methods are described in the Examples below.

The present invention is illustrated in further details by the following non-limiting examples.

### EXAMPLES

### Example 1

### Materials and Methods

Synthesis of [trans-3-hexenoyl]hGHRH (1-44) amide. [trans-3-hexenoyl]hGHRH (1-44) amide is synthesized using FMOC solid phase peptide synthesis starting with Ramage Tricyclic Amide Resin. Protected amino acids and trans-3-hexenoyl acid are used for coupling whereby each protected amino acid and trans-3-hexenoyl acid is dissolved in DMF-treated with aluminum oxide with TBTU to assist in reducing racemization and DIPEA to promote activation before coupling. Completeness of couplings is monitored by the Kaiser ninhydrin test (E. Kaiser et al. Anal. Biochem. “Color Test for Detection of Free Terminal Amino Groups in the Solid Phase Synthesis of Peptides”) and the TNBS test (Means and Feeney, 1971, Holden-Day Inc. San Francisco “Chemical Modification of Proteins” p. 217).

The side chain protecting groups and the peptide-resin bond are cleaved by stirring the protected peptide-resin in a cleavage cocktail consisting of 90% TFA, 5% EDT and 5% water. The crude peptide is purified by HPLC through a three-stage purification scheme using the following buffers, 0.1% MSA, TEAP ph 6.5 and 2% HOAc affording pure [trans-3-hexenoyl]hGHRH (1-44) amide (≧98.5%). The purified peptide lots are pooled and reconstituted in 0.5% acetic acid and lyophilized.

Lyophilization Process. The samples were lyophilized by freezing at −50° C. and holding, annealing to −10° C. and holding, primary drying at −10° C. under 100 mTorr and secondary drying at 25° C. under 100 mTorr.

Formulations. Table I details the constituents of several tested formulations of [trans-3-hexenoyl]hGHRH (1-44) amide as active ingredient. [trans-3-hexenoyl]hGHRH (1-44) amide is present at a concentration of 4 mg/ml in all the formulations listed in Table I except for formulation F12, where the active ingredient is in a concentration of 8 mg/ml.

TABLE I
Constituents of different formulations.

| | | | Bulking agent | Stabilizer (% | | |
|---|---|---|---|---|---|---|
| No. | Buffer | pH | (% w/w) | w/w) | Antioxidant | Surfactant |
| F1 | 10 mM Succinate | 5 | Mannitol (4%) | Trehalose (2%) | none | 0.01% T20 |
| F3 | 10 mM Succinate | 4 | Mannitol (4%) | Trehalose (2%) | none | 0.01% T20 |
| F4 | 10 mM Histidine | 6 | Mannitol (4%) | Trehalose (2%) | none | 0.01% T20 |
| F5 | 10 mM Phosphate | 6 | Mannitol (4%) | Trehalose (2%) | none | 0.01% T20 |
| F6 | 10 mM Succinate | 5 | Glycine (2.5%) | Trehalose (2%) | none | 0.01% T20 |
| F7 | 10 mM Succinate | 5 | PEG 4K (10%) | Trehalose (2%) | none | 0.01% T20 |
| F8 | 10 mM Succinate | 5 | Mannitol (2%) + PEG | Trehalose (2%) | none | 0.01% T20 |
| | | | (5%) | | | |
| F9 | 10 mM Succinate | 5 | Mannitol (4%) | Lactose (2%) | none | 0.01% T20 |
| F10 | 10 mM Succinate | 5 | Mannitol (4%) | Sucrose (2%) | none | 0.01% T20 |
| F11 | 10 mM Succinate | 5 | Mannitol (4%) | Trehalose (2%) | 1 mM | 0.01% T20 |
| | | | | | Methionine | |
| F12 | 10 mM Succinate | 5 | Mannitol (4%) | Trehalose (2%) | none | 0.01% T20 |
| F13 | 10 mM Histidine | 6 | Mannitol (4%) | Sucrose (2%) | none | 0.01% T20 |
| F14 | 10 mM Succinate | 5.5 | Mannitol (4%) | Sucrose (2%) | none | 0.01% T20 |

* T20 means polysorbate-20, and histidine means L-histidine.

### Process for Making the Formulations

The formulations were prepared by combining the ingredients, mixing, and adjusting pH as appropriate. By way of example, details of the preparation of Formulation 13 and 14 (F13 and F14; see Table I) are provided below.

### F13 was Prepared as Follows:

The following stock solutions were prepared:
0.1 M HCL stock solution;
10 mM Histidine stock solution; and
1% Polysorbate 20 stock solution (1 g of Polysorbate 20 was combined with 10 mM Histidine stock solution to reach a volume of 100 mL).

The ingredients were combined as per Table II:

TABLE II
Ingredients - F13

| Ingredient | Quantity per kg | |
|---|---|---|
| Mannitol | 40 | g |
| Sucrose | 20 | g |
| Polysorbate 1% stock solution | 10 | ml |
| Histidine, free base | 1.55 | g |
| [trans-3-hexenoyl]hGHRH | 4 | g |
| (1-44) amide | | |

The ingredients were combined as follows:
The container was filled with 0.90 kg sterile water;
1.55 g of Histidine (free base) was added to the container while mixing, followed by gentle agitation for 10 minutes at ambient temperature;
40 g of Mannitol was added to the container while mixing, followed by gentle agitation for 10 minutes at ambient temperature;
20 g of Sucrose was added to the container while mixing, followed by gentle agitation for 10 minutes at ambient temperature;
10 ml of Polysorbate 1% stock solution was added, followed by gentle agitation for 15 minutes at ambient temperature;
The pH of the formulation was determined and, if necessary, the formulation was titrated to pH 6.6±0.2 using 0.1M HCL stock solution or 10 mM Histidine stock solution as appropriate;
4 g of [trans-3-hexenoyl]hGHRH (1-44) amide was added and the mixture was gently agitated until [trans-3-hexenoyl]hGHRH (1-44) amide was completely dissolved or for at least 30 minutes at ambient temperature;
The pH of the formulation was determined, if necessary, the formulation was titrated to pH 6.0±0.2 using 0.1 M HCl stock solution or 10 mM Histidine stock solution as appropriate;
The solution was brought to 1 kg with sterile water and filtered through a 0.22 μm membrane.

### F14 was Prepared as Follows:

The following stock solutions were prepared:
0.1 M NaOH stock solution;
10 mM sodium succinate stock solution; and
1% Polysorbate 20 stock solution (1 g of Polysorbate 20 was combined with 10 mM sodium succinate stock solution to reach a volume of 100 mL).

Subsequently, the ingredients were combined as per Table III:

TABLE III
Ingredients - F14

| Ingredient | Quantity per kg | |
|---|---|---|
| Mannitol | 40 | g |
| Sucrose | 20 | g |
| Polysorbate 1% stock solution | 10 | ml |
| Sodium succinate | 2.70 | g |
| [trans-3-hexenoyl]hGHRH | 4 | g |
| (1-44) amide | | |

The ingredients were combined as follows:
The container was filled with 0.90 kg sterile water;
2.70 g of sodium succinate was added to the container while mixing, followed by gentle agitation for 10 minutes at ambient temperature;
40 g of Mannitol was added to the container while mixing, followed by gentle agitation for 10 minutes at ambient temperature;
20 g of Sucrose was added to the container while mixing, followed by gentle agitation for 10 minutes at ambient temperature;
10 ml of 1% Polysorbate stock solution was added, followed by gentle agitation for 15 minutes at ambient temperature;
The pH of the formulation was determined and, if necessary, the formulation was titrated to pH 6.1±0.2 using 0.1 M NaOH stock solution or 10 mM sodium succinate stock solution;
4 g of [trans-3-hexenoyl]hGHRH (1-44) was amide added and the mixture was gently agitated until [trans-3-hexenoyl]hGHRH (1-44) amide was completely dissolved, or for at least 30 minutes at ambient temperature;
The pH of the formulation was determined and, if necessary, the formulation was titrated to pH 5.5±0.2 using 0.1 M NaOH stock solution or 10 mM sodium succinate stock solution as appropriate;
The solution was brought to 1 kg with sterile water and filtered through a 0.22 μm membrane.

Reverse Phase high performance liquid chromatography (RP-HPLC). HPLC analysis was performed using the Agilent 1100™ HPLC system, a WATERS DeltaPak™ HPI C18 column, a mobile phase (Acetonitrile/Milli-Q water) at 1.0 ml/min and UV detection at 214 nm.

Identification and Quantification of [trans-3-hexenoyl]hGHRH (1-44) amide Using Reverse Phase HPLC. Identification and quantification of [trans-3-hexenoyl]hGHRH (1-44) amide was established by comparing its retention time in the sample with the respective retention time of freshly prepared calibrated standard [trans-3-hexenoyl]hGHRH (1-44) amide solutions made from [trans-3-hexenoyl]hGHRH (1-44) amide from the same lot. The quantity of [trans-3-hexenoyl]hGHRH (1-44) amide in the samples was calculated by comparison to a standard curve obtained with serial dilutions of known concentrations.

### Example 2

### Results

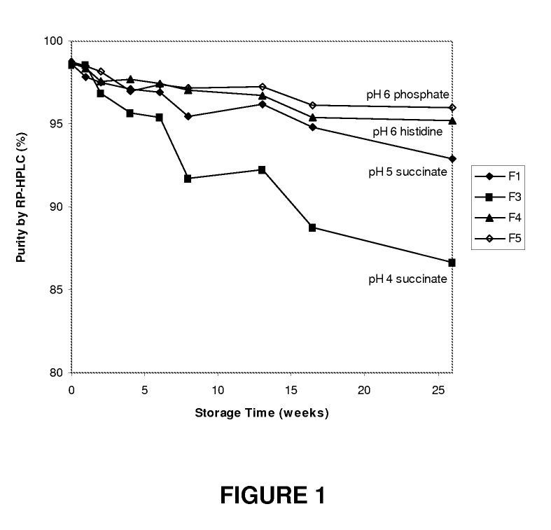

FIG. 1 compares the [trans-3-hexenoyl]hGHRH (1-44) amide purity levels of formulation combinations prepared using three different buffers (phosphate, histidine and succinate) and having three different pH (4.0, 5.0 and 6.0) after storage in a lyophilized form at 40° C. Samples were tested before lyophilization and upon reconstitution after a period of lyophilization at 40° C. of 1 week, 2 weeks, 4 weeks, 6 weeks, 8 weeks, 3 months, 4 months and 6 months. It can be observed that use of phosphate buffer at pH 6.0 (F5), histidine buffer at pH 6.0 (F4) and succinate buffer at pH 5 (F1) results in formulation having a good stability upon storage at 40° C. Succinate buffer at pH 4 (F3) was less stable upon storage at 40° C. but can still provide a suitable formulation.

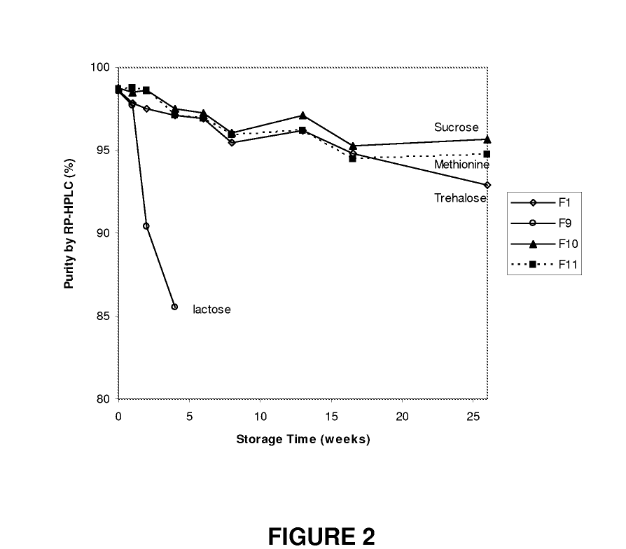

FIG. 2 compares different stabilizers (trehalose, lactose, and sucrose) and the presence of antioxidant (methionine) after storage in a lyophilized form at 40° C. Samples were tested before lyophilization and upon reconstitution after a period of lyophilization at 40° C. of 1 week, 2 weeks, 4 weeks, 6 weeks, 8 weeks, 3 months, 4 months and 6 months. It can be observed that the non-reducing sugars trehalose (F1 and F11) and sucrose (F10) provide a good stabilization at 40° C. The addition of methionine as an antioxidant (F11) has a slight positive impact on stability compared to the same formulation without methionine (F1). In contrast, the reducing sugar lactose (F9) does not provide stabilization at 40° C.

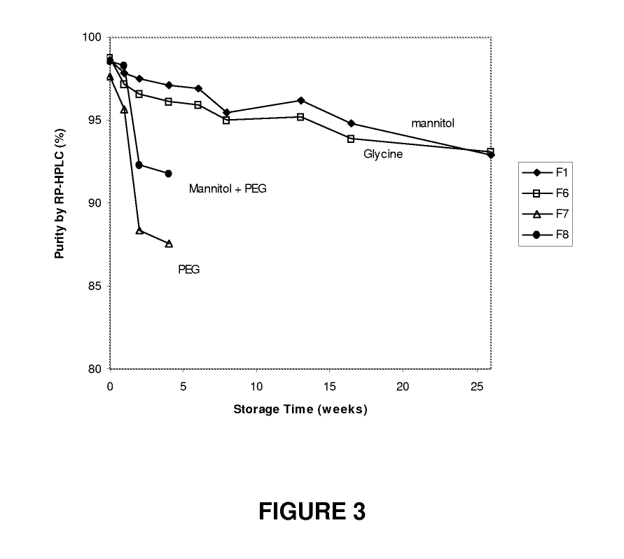

FIG. 3 compares a variety of bulking agents (mannitol, glycine, PEG, mannitol and PEG) after storage in a lyophilized form at 40° C. Samples were tested before lyophilization and upon reconstitution after a period of lyophilization at 40° C. of 1 week, 2 weeks, 4 weeks, 6 weeks, 8 weeks, 3 months, 4 months and 6 months. It can be observed that mannitol (used alone) (F1) and glycine (F6) provide a good stabilization of the formulation after storage at 40° C.

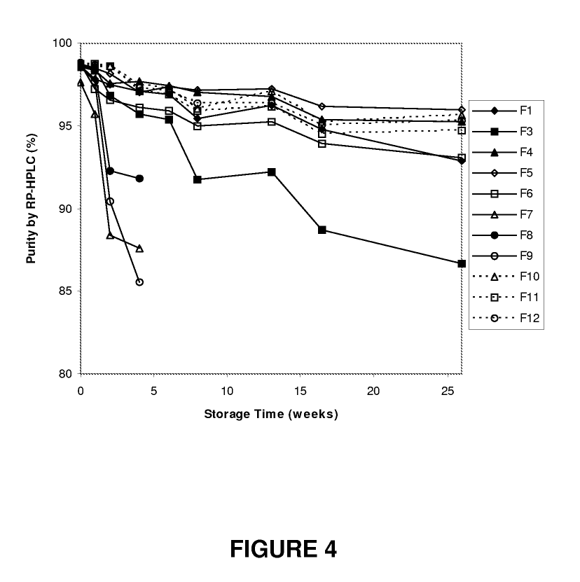

The RP-HPLC results of formulations F1, F3, F4, F5, F6, F7, F8, F9, F10, F11 and F12 are illustrated in FIG. 4 after 1 week, 2 weeks, 4 weeks, 6 weeks, 8 weeks, 3 months, 4 months and 6 months of storage in a lyophilized form at 40° C. After six-month storage at 40° C. which represents a stressful condition, the most stable formulations were formulations F1, F4, F5, F6, F10, F11 and F12.

Based on the results of FIGS. 1, 2, 3 and 4, the combination of constituents corresponding to formulations F13 and F14 have been designed, which are: 4% Mannitol, 2% Sucrose, 0.01% Polysorbate 20 pH 6.0 in Histidine Buffer (F13); and 4% Mannitol, 2% Sucrose, 0.01% Polysorbate 20 pH 5.5 in Sodium Succinate Buffer (F14).

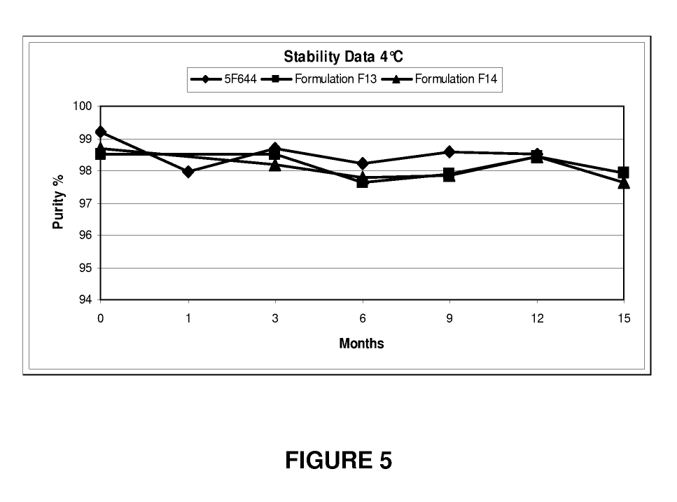

FIG. 5 illustrates the stability profile of formulations F13 and F14 along with a non-stabilized formulation (5F644) in a lyophilized form at 40° C. over a period of 15 months. Reconstitution with sterile water is made just prior to the purity tests. The non-stabilized formulation (5F644) contains 4% mannitol, 1 mg/ml of [trans-3-hexenoyl]hGHRH (1-44) amide and pH 6.0 is obtained with NaOH addition. FIG. 5 shows that the purity profiles are similar for formulations F13 and F14 and the non-stabilized formulation (5F644) at 4° C. All the remaining formulations of Table I have a similar purity profiles than the non-stabilized formulation (5F644) at 4° C. (not shown).

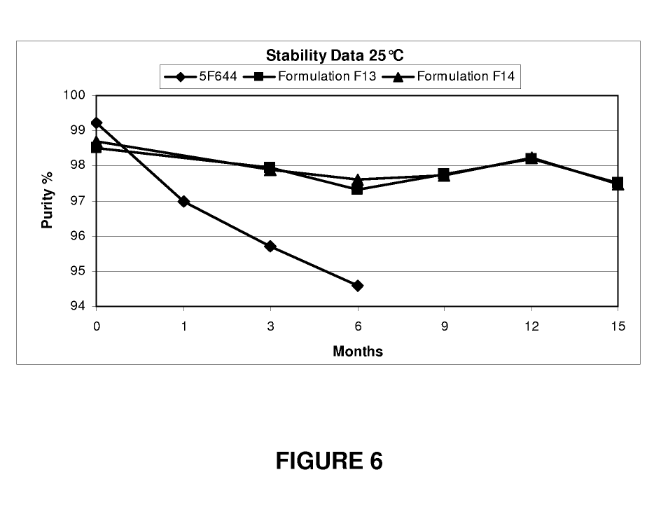

FIG. 6 illustrates the stability profile of formulations F13 and F14 along with the non-stabilized formulation in a lyophilized form at 25° C. over a period of 15 months. Reconstitution with sterile water is made just prior to the purity tests. The non-stabilized formulation (5F644) shows pronounced degradation at 6 months whereas the formulations F13 and F14 remain stable after 15 months.

Moisture content for all lyophilized formulations at two months was advantageously less than 1% by Karl Fisher Moisture Analysis. Karl Fisher Moisture Analysis (KF) is a standard, well known test to determine the water content of a product or composition.

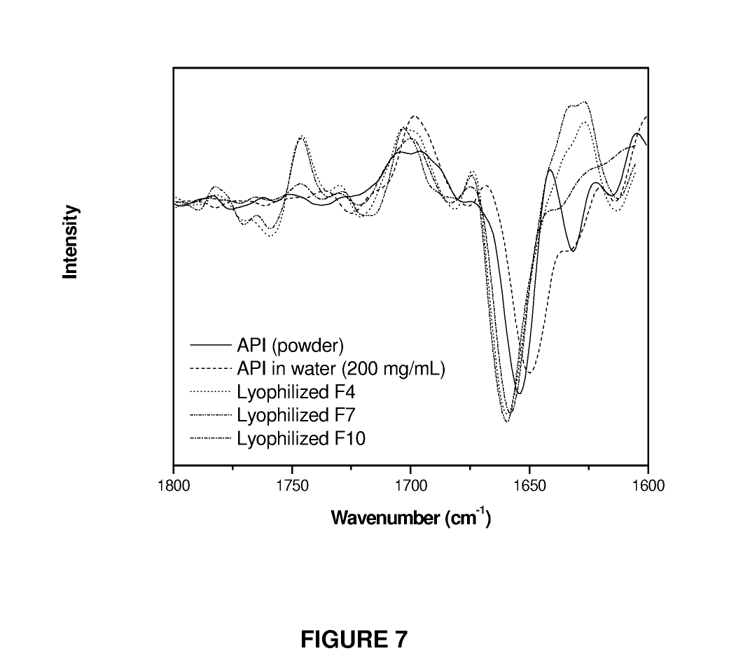

FIG. 7 compares FT/IR analyses of lyophilized samples of the formulations F4, F7 and F10 in comparison with the active principal ingredient (API) alone, in powder form and in liquid form (solubilized with water at 200 mg/ml). In this case, the API is (hexenoyl trans-3)hGHRH(1-44)NH₂. Generally, FT/IR analyses provide information on the secondary structure of the active ingredient. More specifically, FIG. 7 shows that [trans-3-hexenoyl]hGHRH (1-44) amide peptide retains its native structure in the tested formulations. Signals at 1660 cm⁻¹ indicates the conserved α-helical structure of [trans-3-hexenoyl]hGHRH (1-44) amide peptide.

Although the present invention has been described hereinabove by way of specific embodiments thereof, it can be modified, without departing from the spirit and nature of the subject invention as defined in the appended claims.

What is claimed is:

1. A solid pharmaceutical formulation comprising:
a GHRH molecule,
an anionic surfactant, and
a non-reducing sugar,
wherein the GHRH molecule is a GHRH analog of formula A:
X-GHRH Peptide (A)
wherein;
the GHRH peptide is a peptide of formula B:
(SEQ ID NO: 1)
A1-A2-Asp-Ala-Ile-Phe-Thr-A8-Ser-Tyr-Arg-Lys-A13-
Leu-A15-Gln-Leu-A18-Ala-Arg-Lys-Leu-Leu-A24-A25-
Ile-A27-A28-Arg-A30-R0 (B)
wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)n-CONH₂, with n=1 to 12; and
X is a hydrophobic tail anchored via an amide bond to the N-terminus of the peptide and the hydrophobic tail defining a backbone of 5 to 7 atoms;
wherein the backbone can be substituted by C₁₋₆ alkyl, C₃₋₆ cycloalkyl, or C₆₋₁₂ aryl and the backbone comprises at least one rigidifying moiety connected to at least two atoms of the backbone;
said moiety is a double bond, triple bond, saturated or unsaturated C₃₋₉ cycloalkyl, or C₆₋₁₂ aryl.

2. The formulation of claim 1, wherein X is:

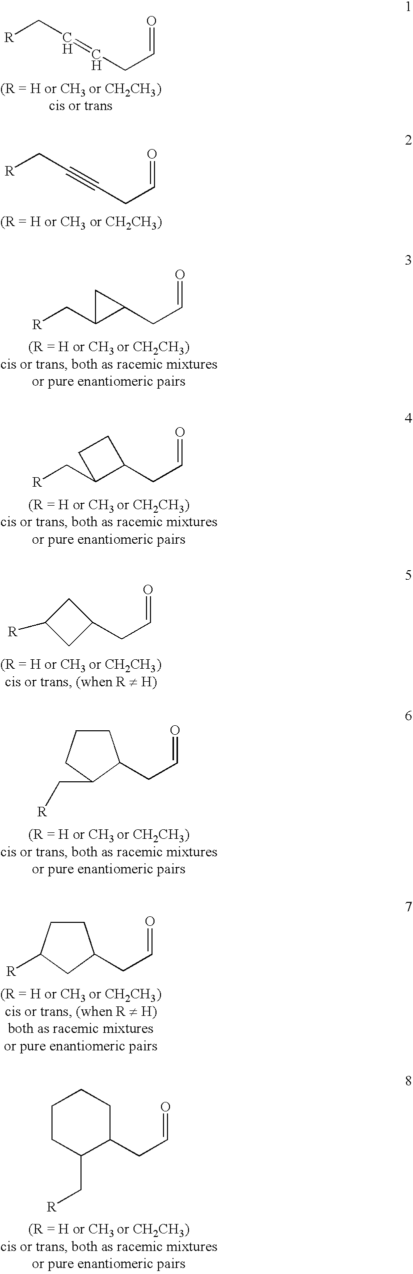

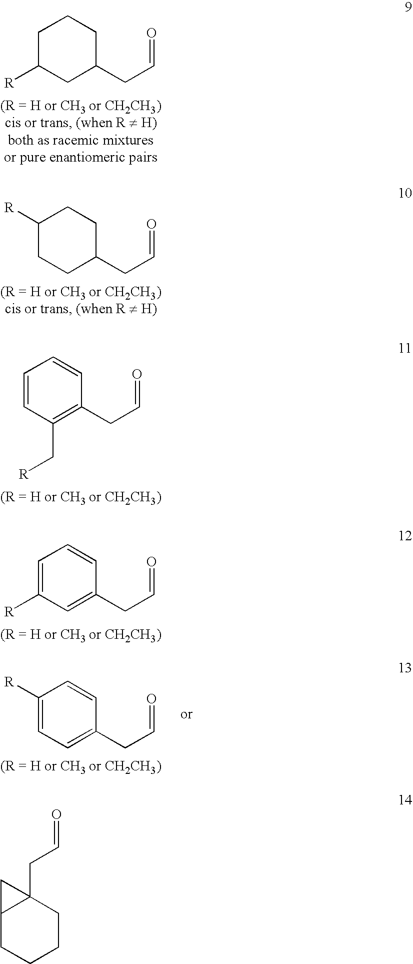

3. The formulation of claim 1, wherein A30 is:
(a) a bond;
(b) an amino acid sequence corresponding to positions 30-44 of a natural GHRH peptide (SEQ ID NO: 6); and
(c) said amino acid sequence of SEQ ID NO: 6, having a 1-14 amino acid deletion from its C-terminus.

4. The formulation of claim 1, wherein said GHRH peptide is:
(a) a polypeptide comprising the amino acid sequence of SEQ ID NO: 2 or 3;
(b) a polypeptide comprising the amino acid sequence of SEQ ID NO: 4 or 5; or
(c) said polypeptide of (a) having a 1 to 14 amino acid deletion from its C-terminus.

5. The formulation of claim 1, wherein the GHRH analog is (hexenoyl trans-3)hGHRH(1-44)NH₂ (SEQ ID NO: 7).

6. The formulation of claim 1, wherein the formulation is lyophilized.

7. The formulation of claim 1, wherein the non-reducing sugar is trehalose or sucrose.

8. The formulation of claim 7, wherein the non-reducing sugar is trehalose.

9. The formulation of claim 7, wherein the non-reducing sugar is sucrose.

10. The formulation of claim 1, wherein the anionic surfactant is a polyoxyethylene sorbitan alkyl ester.

11. The formulation of claim 10, wherein the anionic surfactant is polysorbate-20.

12. The formulation of claim 1, having a pH of about 4.0 to about 7.5 when measured upon suspension in water.

13. The formulation of claim 1, wherein the formulation further comprises a bulking agent.

14. The formulation of claim 13, wherein the bulking agent is mannitol.

15. The formulation of claim 1, wherein the formulation further comprises an anti-oxidant agent.

16. The formulation of claim 15, wherein the anti-oxidant agent is methionine.

17. A liquid pharmaceutical formulation comprising:
a GHRH molecule,
an anionic surfactant, and
a non-reducing sugar,
wherein said formulation has a pH of about 4.0 to about 7.5,
wherein the GHRH molecule is a GHRH analog of formula A:
X-GHRH Peptide (A)
wherein;
the GHRH peptide is a peptide of formula B:
(SEQ ID NO: 1)
A1-A2-Asp-Ala-Ile-Phe-Thr-A8-Ser-Tyr-Arg-Lys-A13-
Leu-A15-Gln-Leu-A18-Ala-Arg-Lys-Leu-Leu-A24-A25-
Ile-A27-A28-Arg-A30-R0 (B)
wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)n-CONH₂, with n=1 to 12; and
X is a hydrophobic tail anchored via an amide bond to the N-terminus of the peptide and the hydrophobic tail defining a backbone of 5 to 7 atoms;
wherein the backbone can be substituted by C₁₋₆ alkyl, C₃₋₆ cycloalkyl, or C₆₋₁₂ aryl and the backbone comprises at least one rigidifying moiety connected to at least two atoms of the backbone;
said moiety is a double bond, triple bond, saturated or unsaturated C₃₋₉ cycloalkyl, or C₆₋₁₂ aryl.

18. The formulation of claim 17, wherein X is:

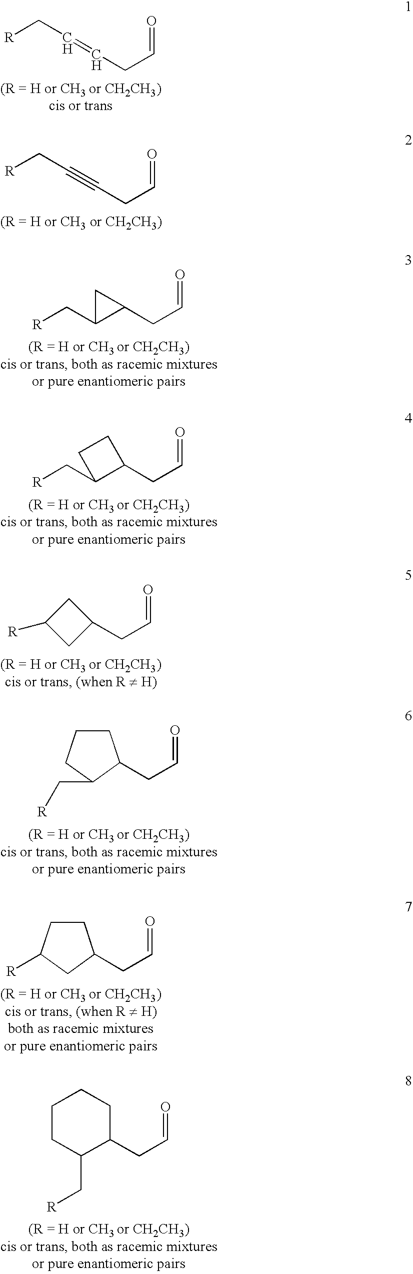

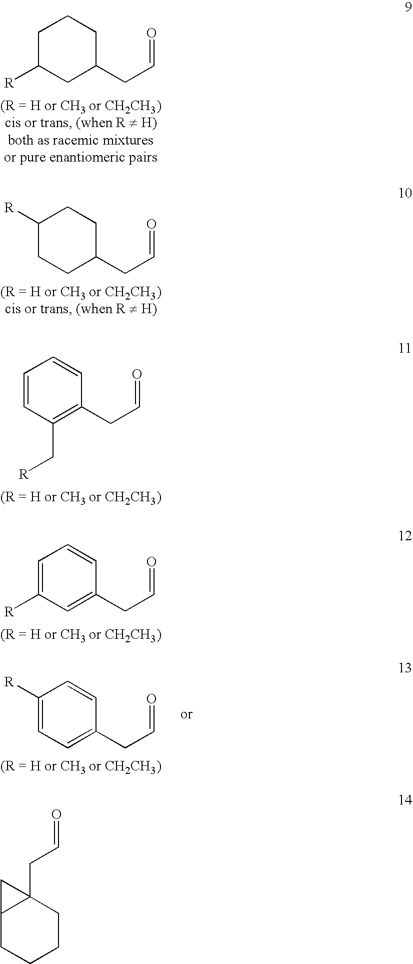

19. The formulation of claim 17, wherein A30 is:
(a) a bond;
(b) an amino acid sequence corresponding to positions 30-44 of a natural GHRH peptide (SEQ ID NO: 6), or
(c) said SEQ ID NO: 6 having a 1-14 amino acid deletion from its C-terminus.

20. The formulation of claim 17, wherein said GHRH peptide is:
(a) a polypeptide comprising the amino acid sequence of SEQ ID NO: 2 or 3;
(b) a polypeptide comprising the amino acid sequence of SEQ ID NO: 4 or 5; or
(c) said polypeptide of (a) having a 1 to 14 amino acid deletion from its C-terminus.

21. The formulation of claim 17, wherein the GHRH analog is (hexenoyl trans-3)hGHRH(1-44)NH₂ (SEQ ID NO: 7).

22. The formulation of claim 17, wherein the non-reducing sugar is trehalose or sucrose.

23. The formulation of claim 22, wherein the non-reducing sugar is trehalose.

24. The formulation of claim 22, wherein the non-reducing sugar is sucrose.

25. The formulation of claim 17, wherein the non-reducing sugar is present in a concentration of about 0.1 to about 5% (w/w).

26. The formulation of claim 25, wherein the non-reducing sugar is present in a concentration of about 2% (w/w).

27. The formulation of claim 17, wherein the anionic surfactant is a polyoxyethylene sorbitan alkyl ester.

28. The formulation of claim 27, wherein the anionic surfactant is polysorbate-20.

29. The formulation of claim 17, wherein the surfactant is present in a concentration of about 0.001% (w/w) to about 0.1% (w/w).

30. The formulation of claim 29, wherein the surfactant is present at a concentration of about 0.01% (w/w).

31. The formulation of claim 17, having a pH of about 5.0 to about 6.0.

32. The formulation according to claim 31, having a pH of about 5.0.

33. The formulation according to claim 31, having a pH of about 5.5.

34. The formulation according to claim 31, having a pH of about 6.0.

35. The formulation of claim 17, further comprising a buffer, wherein said buffer is (i) succinate buffer, (ii) histidine buffer, (iii) phosphate buffer or (iv) any combination of (i) to (iii).

36. The formulation of claim 17, wherein the formulation further comprises a bulking agent.

37. The formulation of claim 36, wherein the bulking agent is mannitol.

38. The formulation of claim 36, wherein the bulking agent is present in an amount of about 1 to about 10% (w/w).

39. The formulation according to claim 38, wherein the bulking agent is present in an amount of about 4% (w/w).

40. The formulation of claim 17, wherein the formulation further comprises an anti-oxidant agent.

41. The formulation of claim 40, wherein the anti-oxidant agent is methionine.

42. The formulation of claim 17, comprising:
[trans-3-hexenoyl]hGHRH (1-44) amide,
about 0.01% (w/w) of polysorbate-20,
about 2% (w/w) of (i) trehalose, (ii) sucrose or (iii) any combination of (i) and (ii),
about 4% (w/w) of mannitol; and
(i) succinate buffer, (ii) histidine buffer or (iii) any combination of (i) and (ii),
said formulation having a pH of about 5.0 to about 6.0.

43. The formulation of claim 17, comprising:
[trans-3-hexenoyl]hGHRH (1-44) amide,
about 0.01% (w/w) of polysorbate-20,
about 2% (w/w) sucrose,
about 4% (w/w) mannitol, and
an histidine buffer,
said formulation having a pH of about 6.0.

44. The formulation of claim 17, comprising:
[trans-3-hexenoyl]hGHRH (1-44) amide,
about 0.01% (w/w) polysorbate-20,
about 2% (w/w) sucrose,
about 4% (w/w) mannitol, and
a succinate buffer,
said formulation having a pH of about 5.5.

45. The formulation of claim 17, comprising:
[trans-3-hexenoyl]hGHRH (1-44) amide,
about 0.01% (w/w) polysorbate-20,
about 2% (w/w) sucrose,
about 4% (w/w) mannitol, and
a succinate buffer,
said formulation having a pH of about 5.0.

46. The formulation of claim 17, comprising:
[trans-3-hexenoyl]hGHRH (1-44) amide,
about 0.01% (w/w) polysorbate-20,
about 2% (w/w) trehalose,
about 4% (w/w) mannitol, and
a succinate buffer,
said formulation having a pH of about 5.5.

47. A lyophilized pharmaceutical formulation prepared by lyophilizing the liquid formulation of claim 17.

48. A lyophilized pharmaceutical formulation prepared by lyophilizing the liquid formulation of claim 21.

49. A lyophilized pharmaceutical formulation prepared by lyophilizing the liquid formulation of claim 42.

50. A lyophilized pharmaceutical formulation prepared by lyophilizing the liquid formulation of claim 43.

51. A lyophilized pharmaceutical formulation prepared by lyophilizing the liquid formulation of claim 44.

52. A lyophilized pharmaceutical formulation prepared by lyophilizing the liquid formulation of claim 45.

53. A lyophilized pharmaceutical formulation prepared by lyophilizing the liquid formulation of claim 46.

54. A method of treating at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease in a subject, comprising administering the liquid pharmaceutical formulation of claim 17 to said subject.

55. A method of treating at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease in a subject, comprising administering a liquid pharmaceutical formulation prepared by the suspension of the solid pharmaceutical formulation of claim 1 with a sterile aqueous solution to said subject.

56. A method of treating at least one of HIV-associated lipodystrophy, HIV-lipohypertrophy, abdominal obesity, GH deficiency, frailty, mild cognitive impairment, immune deficiency, wasting associated with a chronic disease or long-term disease, or malnutrition associated with a chronic disease or long-term disease in a subject, comprising administering a liquid pharmaceutical formulation prepared by the suspension of the lyophilized pharmaceutical formulation of claim 47 with a sterile aqueous solution to said subject.

57. The method of claim 55, wherein the administration is via a subcutaneous, intramuscular, intravenous or intraperitoneal route.

58. The method of claim 56, wherein the administration is via a subcutaneous, intramuscular, intravenous or intraperitoneal route.

59. The method of claim 57, wherein the administration is via a subcutaneous, intramuscular, intravenous or intraperitoneal route.


SEQUENCE LISTING (7 sequences)

SEQ ID NO: 1 - protein, 44 aa, Artificial sequence
  GRF peptide
XXDAIFTXSYRKXLXQLXARKLLXXIXXRXXXXXXXXXXXXXXX

SEQ ID NO: 2 - protein, 44 aa, Homo sapiens
  MISC_FEATURE (44)..(44): Leu residue is capped with an unsubstituted amide moiety
YADAIFTNSYRKVLGQLSARKLLQDIMSRQQGESNQERGARARL

SEQ ID NO: 3 - protein, 44 aa, Artificial sequence; identical to SEQ ID NO: 2
  Amino acid sequence of human GRF

SEQ ID NO: 4 - protein, 29 aa, Homo sapiens
  MISC_FEATURE (29)..(29): Arg residue is capped with an unsubstituted amide moiety
YADAIFTNSYRKVLGQLSARKLLQDIMSR

SEQ ID NO: 5 - protein, 29 aa, Artificial sequence
  Amino acid sequence of minimum active core of human GRF
YADAIFTNSYRKVLGQLSARKLLQDIMSR

SEQ ID NO: 6 - protein, 15 aa, Artificial sequence
  Amino acid sequence corresponding to positions 30 to 44 of humanGRF
QQGESNQERGARARL

SEQ ID NO: 7 - protein, 44 aa, Artificial sequence; identical to SEQ ID NO: 2
  Modified GRF peptide